(19) United States
(12) Patent Application Publication
(10) Pub. No.: US 2015/0057305 A1
(43) Pub. Date: Feb. 26, 2015

(54) PHARMACEUTICAL COMPOSITION

(71) Applicant: SHANGHAI HOTMED SCIENCES CO., LTD., Yangpu District, Shanghai (CN)

(72) Inventors: Kaiyong Tang, Yangpu District (CN); Junfang Pan, Yangpu District (CN); Qin Zhu, Yangpu District (CN); Tong Fang, Yangpu District (CN); Chengcheng Niu, Yangpu District (CN); Wanhai Li, Yangpu District (CN); Wei Lv, Yangpu District (CN); Chen Qing, Yangpu District (CN); Guorong Fan, Yangpu District (CN)

(21) Appl. No.: 14/389,468

(22) PCT Filed: Mar. 12, 2013
(86) PCT No.: PCT/CN2013/072492

§ 371 Date: Sep. 30, 2014

(30) Foreign Application Priority Data
Apr. 1, 2012 (CN) 201210093955.7

(51) Int. Cl. A61K 47/40; A61K 31/4745

(52) U.S. Cl. CPC A61K 47/40; A61K 31/4745
USPC 514/283

(57) ABSTRACT

Disclosed in the present invention is a pharmaceutical composition, comprising a weight ratio of 1:120 to 1:1000 of camptothecin compound of formula I and β-cyclodextrin or derivatives thereof, and an acidic buffer to adjust the pH=3.5-6.0. The composition can be used to treat solid tumours, such as melanoma, pancreatic cancer, hepatoma etc. The pharmaceutical composition of the present invention is miscible with a water-miscible co-solvent system in any proportion, and can be used as an intravenous infusion solvent, and has no obvious hemolysis or vascular stimulation; the pharmaceutical composition has a better tumour inhibiting rate than solubilisation of surfactants.

### FIELD OF THE INVENTION

The present invention belongs to the field of medicine technology, and relates to a pharmaceutical composition, more particularly to a pharmaceutical composition comprising camptothecin compound of formula I and β-cyclodextrin or derivatives thereof for treating solid tumours.

### BACKGROUND OF THE INVENTION

The china patent application CN 1897942A disclosed that: the aqueous solubility of drug substances plays an important role in the formulation of drug dosage forms. For the oral route of administration it is well experienced that, unless the substance has an aqueous solubility above 10 mg/ml over the pH-range 1-7, then potential absorption problems may occur. A solubility less than 1 mg/ml is likely to give dissolution-rate limited absorption because solubility and dissolution rate are interrelated.

Camptothecin anticancer compounds are basically administrated by intravenous infusion, and also have small oral preparations such as topotecan hydrochloride capsules [Hycamtin, 1 mg×10 capsules/box, or 0.25 mg×10 capsules/box, Glaxo Smith Kline produced]. Current studies suggest that the anticancer effect of the camptothecin anticancer compounds mainly depend on the open-closed-loop of the E-ring, and develop anticancer activity at the closed state of the E-ring. Since the E-ring of the camptothecin anticancer compounds is present in the closed structure in acidic condition, they are more suitable for oral administration.

However it is well known that, most of camptothecin compounds have low water soluble, for example 9-nitro-camptothecin, camptothecin, 9-amino-camptothecin etc., and do not dissolve in water, and also difficult to dissolve in many kinds of mineral oil, have poor stability, therefore they are not suitable for preparing injection. YAN Jing-chao etc. studied the pharmacokinetics of 9-nitro-camptothecin (9-NC) in dogs, from the results found that the oral bioavailability of 9-NC is poor (less than 6%) [YAN Jing-chao etc., Chin J Clin Pharmacol Ther 2005 November; 10(11)].

Therefore, development of a novelty of camptothecin compound solution preparation has become a major problem for many researchers for now, while only the United States SuperGen company has developed 9-nitro-camptothecin for oral administration.

### SUMMARY OF THE INVENTION

The technical problem to be solved by the present invention is to provide a pharmaceutical composition comprising camptothecin compound of formula I for treating of solid tumors such as melanoma, pancreatic cancer, hepatoma etc., which shows good safety, convenience, effectiveness and dose characteristics.

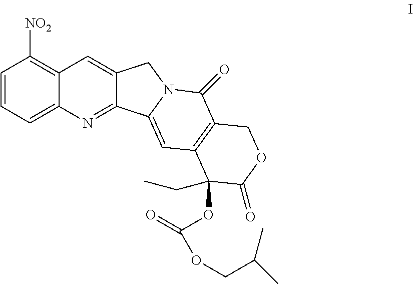

Wherein, the above-mentioned camptothecin compound of formula I (hereinafter referred to as the compound of formula I) is disclosed in CN 100363366C for the treating solid tumors. The compound stabilized the lactone-ring structure of the camptothecin by introducing carbonate, to improve the stability and the antitumor activity in vivo and reduce side effects. The pharmacological experiments demonstrate that the compound of formula I has good stability and antitumor activity in vivo in human plasma, I.p administrating, the tumor inhibition rate for S-180 tumors is 20.1˜92.2%.

According to the GB/T 21853-2008 chemicals distribution coefficient (n-octanol-water) shake flask method, from the experiments results it can be seen that the compound of formula I almost insoluble in water (Lg Po/w is 7.31 in water), and also difficult to dissolve in many kinds of mineral oil, the solution stability is poor.

Therefore, with regards to the water-insoluble compound of formula I, a water soluble β-cyclodextrin or derivatives thereof is added thereto for solubilising and improving the defects of the compound of formula I water-insoluble or slightly soluble in water, so as to achieve the desired dosage of anticancer activity, and which can be miscible with a water-miscible co-solvent system in any proportion, has no obvious hemolysis and vascular stimulation as an intravenous infusion solvent.

For the purpose of the above-mentioned object, the present invention employs the following technical solutions to achieve.

With regards to the pharmaceutical composition, it is a pharmaceutical composition in aqueous solution form or in solid form, comprising a weight ratio of 1:120 to 1:1000 of the compound of formula I and β-cyclodextrin or derivatives thereof, and an acidic buffer to adjust pH=3.5˜6.0.

The weight ratio between the compound of formula I and β-cyclodextrin or derivatives thereof is preferably 1:200 to 1:800. And according to the specific conditions, different types of the β-cyclodextrin require different ratios to inhibit or prevent the compound of formula I precipitation.

The β-cyclodextrin or derivatives thereof are selected from the group consisting of hydroxypropyl-β-cyclodextrin and/or sulfobutyl ether sodium β-cyclodextrin.

The acidic buffer is selected from the group consisting of tartaric acid or salts thereof, citric acid or salts thereof, hydrochloric acid or salts thereof, acetic acid or salts thereof, maleic acid or salts thereof, malic acid or salts thereof, sulphuric acid or salts thereof, phosphoric acid or salts thereof, lactic acid or salts thereof etc. The amount of the acidic butter is to adjust the pH of the pharmaceutical composition in aqueous solution form in the range of 3.5˜5.5, or adjust the pH of the pharmaceutical composition in solid form in the range of 3.5˜6.0.

A method for preparing the pharmaceutical composition of the present invention including the following steps:

a water-soluble β-cyclodextrin or derivatives thereof is dissolved in water, adding an acidic butter to adjust pH in the range of 3.5 to 6.0 (preferably 4.0); then the compound of formula I is added with stirring to dissolve; adding medicinal activated C, stirring for adsorption and decarburization; and then water was added to the full amount, sterilization filtration, spray drying, or freeze drying after split charging, to give the pharmaceutical composition, or directly split charging.

Wherein the compound of formula I and the water-soluble β-cyclodextrin or derivatives thereof is formed to an inclusion complex, the inclusion complex may be a solid complex by the formation of spray drying or freeze drying, or be an aqueous solution, which could be miscible with a water-miscible co-solvent system in any proportion. The solid complex may added water to form an injection, and also may be a physical mixture of the compound of formula I and the water-soluble β-cyclodextrin or derivatives thereof.

The pharmaceutical compositions of the present invention (using the water-soluble β-cyclodextrin or derivatives thereof to solubilize) and the concentrated solution of the compound of formula I (using surfactants to solubilize) are performed sarcoma inhibition test, the results indicate that the tumour inhibition rate of the pharmaceutical compositions of the present invention by the means of the cyclodextrin for solubilizing higher than that of the compound of formula I by the means of surfactants for solubilizing (especially for sarcoma S180 and hepatoma H22), and have no obvious hemolysis, vascular stimulation and anaphylactic reaction, the side effects also is less than that of the concentrated solution of the compound of formula I. Thus the pharmaceutical compositions according to the present invention are suitable for the treatment of solid tumours for patients.

### DETAILED DESCRIPTION OF EMBODIMENTS OF THE INVENTION

For the purpose of describing the present invention in more detail, the present invention will be described below using examples. These examples are merely for illustrating the present invention, and not intended to limit the present invention.

### EXAMPLES

### Example 1

### Preparation of the Pharmaceutical Composition

1) Formula:

| | | |
|---|---|---|
| The compound of formula I | 1 | g |
| Hydroxypropyl-β-cyclodextrin | 800 | g |
| Added water to | 2000 | ml |

2): Preparation:

Water for injection is added into 800 g of hydroxypropyl-3-cyclodextrin (MS 4.1˜4.8) with stirring and dissolved to achieve 80% of the total volume; hydrochloric acid is added to adjust pH=3.5˜4.5; the compound of formula I is added with stirring to dissolve; added medicinal activated C with stirring for adsorption and decarburization; added water to the full volume, sterilization filtration, spray drying, or freeze drying after split charging, to give the pharmaceutical composition, or directly split charging.

### Example 2

### Preparation of the Pharmaceutical Composition

1) Formula:

| | | |
|---|---|---|
| The compound of formula I | 1 | g |
| Sulfobutyl ether sodium β-cyclodextrin | 800 | g |
| Added water to | 2000 | ml |

2): Preparation:

Water for injection is added into 800 g of hydroxypropyl-β-cyclodextrin (MS 4.1˜4.8) with stirring and dissolved to achieve 80% the total volume; hydrochloric acid is added to adjust pH=3.5˜4.5; the compound of formula I is added with stirring to dissolve; added medicinal activated C with stirring for adsorption and decarburization; added water to the full volume, sterilization filtration, spray drying, or freeze drying after split charging, to give the pharmaceutical composition, or directly split charging.

### Example 3

### Preparation of the Pharmaceutical Composition

1) Formula:

| | | |
|---|---|---|
| The compound of formula I | 1 | g |
| Sulfobutyl ether sodium β-cyclodextrin | 400 | g |
| Hydroxypropyl-β-cyclodextrin | 400 | g |
| Added water to | 2000 | ml |

2): Preparation:

Water for injection is added into 400 g of hydroxypropyl-β-cyclodextrin (MS 4.1˜4.8) and 400 g of hydroxypropyl-β-cyclodextrin (MS 4.1˜4.8) with stirring and dissolved to achieve 80% of the total volume; hydrochloric acid is added to adjust pH=3.5˜4.5; the compound of formula I is added with stirring to dissolve; added medicinal activated C with stirring for adsorption and decarburization; added water to the full volume, sterilization filtration, spray drying or freeze drying after split charging, to give the pharmaceutical composition, or directly split charging.

### Example 4

### Preparation of the Control Composition

1) Formula:

| | | |
|---|---|---|
| Hydroxypropyl-β-cyclodextrin | 800 | g |
| Added water to | 2000 | ml |

2): Preparation:

Water for injection is added into 800 g of hydroxypropyl-β-cyclodextrin (MS 4.1˜4.8) with stirring and dissolved to achieve 80% of the total volume; hydrochloric acid is added to adjust pH=3.5˜4.5; added medicinal activated C with stirring for adsorption and decarburization; added water to the full volume, sterilization filtration, spray drying or freeze drying after split charging, to give the control composition, or directly split charging.

### Example 5

### Preparation of the Concentrated Solution of the Compound of Formula I (That is the Surfactant Hydrotrope of the Compound of Formula I)

1) Formula:

| | | |
|---|---|---|
| The compound of formula I | 2 | g |
| Polyethylene glycol 400 | 1000 | g |
| Tween-80 | 300 | g |
| Glacial acetic acid | 20 | ml |
| Added ethanol to | 2000 | ml |

2): Preparation:

The compound of formula I is added into polyethylene glycol 400 with stirring to dissolve, Tween-80 and glacial acetic acid is added into with stirring evenly, and then added ethanol to the total volume, sterilization filtration, filling.

### Example 6

### Preparation of the Control Concentrated Solution

1) Formula:

| | | |
|---|---|---|
| Polyethylene glycol 400 | 1000 | g |
| Tween-80 | 300 | g |
| Glacial acetic acid | 20 | ml |
| Added ethanol to | 2000 | ml |

2): Preparation:

Polyethylene glycol 400, Tween-80 and glacial acetic acid are mixed uniformity, and then added ethanol to the total volume, sterilization filtration, filling.

### Example 7

### Intravenous Injection for Mouse Tumor Inhibition Experiment

The samples prepared by Examples 1 to 3 were diluted with normal saline (NS) to the dosage for administration before using, and formulated the control concentrated solution, the concentrated solution of the compound of formula I, the control composition, the negative control NS as the same method. Use of tail intravenous administrating 0.2 ml (for 10 min), 1 time each day, continuous administration for 10 days, and then done the growth inhibition experiment for Kunming type of mice sarcoma S180 and mice hepatoma H22. The results are shown in table 1 and table 2.

The tumor inhibition rate %=[(the negative tumor weight−the treating tumor weight)/the negative tumor weight]×100

TABLE 1
Growth inhibition experiment for mouse sarcoma S180 (tail intravenous adminstration)

| | | Mouse | | Weight (g) | | | Tumor inhibition |
|---|---|---|---|---|---|---|---|
| | | Before | After | Before | After | Tumor weight | rate |
| Group | Dosage | experiment | experiment | experiment | experiment | (g) | (%) |
| the Negative control (NS) | 0.1 ml/10 kg | 8 | 8 | 20.40 ± 1.31 | 30.60 ± 2.45 | 2.51 ± 0.69 | — |
| the control concentrated | 0.1 ml/10 kg | 8 | 8 | 20.34 ± 1.21 | 29.80 ± 3.15 | 2.34 ± 0.57 | 6.71 |
| solution | | | | | | | |
| the concentrated solution of | 1.5 mg/kg | 8 | 8 | 20.25 ± 1.52 | 28.02 ± 2.19 | 1.48 ± 0.31 | 40.86 |
| the compound of foimula I | | | | | | | |
| the control composition | 0.1 ml/10 kg | 8 | 8 | 20.58 ± 1.33 | 29.37 ± 1.67 | 2.05 ± 0.84 | 18.32 |
| the pharmaceutical | 1 mg/kg | 8 | 8 | 20.69 ± 1.67 | 29.10 ± 3.07 | 1.39 ± 0.67 | 44.39 |
| compositions of the present | 1.5 mg/kg | 8 | 8 | 20.20 ± 1.58 | 28.48 ± 3.40 | 0.99 ± 0.29 | 60.55 |
| invention | 2 mg/kg | 8 | 8 | 20.45 ± 1.70 | 25.09 ± 2.24 | 0.59 ± 0.24 | 76.15 |

TABLE 2
Growth inhibition experiment for mouse hepatoma H22 (tail intravenous adminstration)

| | | Mouse | | Weight (g) | | | Tumor |
|---|---|---|---|---|---|---|---|
| | | Before | After | Before | After | Tumor weight | inhibition rate |
| Group | Dosage | experiment | experiment | experiment | experiment | (g) | (%) |
| the Negative control (NS) | 0.1 ml/10 kg | 7 | 7 | 21.17 ± 1.38 | 32.62 ± 2.31 | 2.03 ± 0.46 | — |
| the control concentrated solution | 0.1 ml/10 kg | 7 | 7 | 20.78 ± 1.38 | 33.15 ± 2.37 | 1.95 ± 0.62 | 5.21 |
| the concentrated solution of | 2 mg/kg | 7 | 7 | 20.98 ± 1.53 | 32.46 ± 2.76 | 1.70 ± 0.43 | 17.29 |
| the compound of formula I | | | | | | | |
| the control composition | 0.1 ml/10 kg | 7 | 7 | 20.67 ± 1.46 | 33.28 ± 0.82 | 2.09 ± 0.44 | −2.70 |
| the pharmaceutical compositions | 2 mg/kg | 7 | 7 | 21.04 ± 1.15 | 25.64 ± 1.52 | 0.14 ± 0.06 | 92.77 |
| of the present invention | 4 mg/kg | 7 | 7 | 21.64 ± 1.15 | 22.35 ± 1.82 | 0.04 ± 0.03 | 97.68 |

From the above experiments results it can be seen that, the concentrated solution of the compound of formula I and the pharmaceutical compositions of the present invention have a certain of tumor inhibition for mice sarcoma S180 and mice hepatoma H22, compared with the negative control NS. But at the same dosage, the tumor inhibition rate of the pharmaceutical compositions of the present invention much higher than that of the concentrated solution of the compound of formula I.

### Example 8

### Growth Inhibition Experiment for Mice by Intragastric Administration

The samples prepared by Examples 1 to 3 were diluted with normal saline (NS) to the dosage for administration before using, and formulated the control concentrated solution, the concentrated solution of the compound of formula I, the control composition, the negative control NS as the same method. By intragastric administration, 1 time each day, continuous administrating for 10 days, and then done the growth inhibition experiment for Kunming type of mice hepatoma H22. The results are shown in table 3.

TABLE 3
Growth inhibition experiment for mice hepatoma H22 (intragastric administration)

| | | Mouse | | Weight (g) | | | Tumor |
|---|---|---|---|---|---|---|---|
| | | Before | After | Before | After | Tumor weight | inhibition rate |
| Group | Dosage | experiment | experiment | experiment | experiment | (g) | (%) |
| the negative control (NS) | 0.1 ml/10 kg | 7 | 7 | 20.91 ± 1.77 | 32.98 ± 3.66 | 2.61 ± 0.54 | — |
| the control concentrated | 0.1 ml/10 kg | 7 | 7 | 20.49 ± 1.88 | 31.74 ± 3.48 | 1.83 ± 0.20 | 29.59 |
| solution | | | | | | | |
| the concentrated solution of | 1.5 mg/kg | 7 | 7 | 20.04 ± 1.35 | 29.66 ± 2.18 | 1.32 ± 0.22 | 40.17 |
| the compound of formula I | | | | | | | |
| the control composition | 0.1 ml/10 kg | 7 | 7 | 20.65 ± 2.00 | 30.90 ± 3.08 | 1.74 ± 0.92 | 33.26 |
| the pharmaceutical | 1 mg/kg | 7 | 7 | 20.13 ± 1.87 | 30.70 ± 2.80 | 1.76 ± 0.34 | 32.43 |
| compositions of the present | 1.5 mg/kg | 7 | 7 | 19.96 ± 2.17 | 32.82 ± 2.63 | 1.25 ± 0.45 | 51.80 |
| invention | 2 mg/kg | 7 | 7 | 20.47 ± 1.65 | 32.42 ± 1.86 | 1.17 ± 0.25 | 54.92 |

The results indicate that the concentrated solution of the compound of formula I and the pharmaceutical compositions of the present invention have a certain of tumor inhibition for mice hepatoma H22 by intragastric administration, compared with the negative control NS. But at the same dosage, the tumor inhibition rate of the pharmaceutical compositions of the present invention is much higher than that of the concentrated solution of the compound of formula I.

### Example 9

### Hemolysis and Vascular Stimulation Experiments

Take the pharmaceutical compositions of the present invention, studies on the anaphylaxis, hemolysis and vascular stimulation for systemic administration, according to <<chemical drug stimulation, anaphylaxis and hemolysis research technical guidelines>>, the negative control NS as comparison. The results indicate that the pharmaceutical compositions of the present invention have no hemolysis, vascular stimulation and allergic reaction for systemic administration.

Finally it should be noted that, the above embodiments are merely to illustrate the technical solutions of the present invention, it is not intended to be limited, although the preferred examples with reference to the present invention have been described in detail, the person skilled in the art should be understood that the present invention may be modification or equivalent replacement, without departing from the spirit and scope of the present invention, and all such modifications and equivalent replacement are believed to be within the spirit and scope of the invention as defined by the claims of the present invention.

What is claimed is:

1. A pharmaceutical composition comprising a weight ratio of 1:120 to 1:1000 of camptothecin compound of formula I and β-cyclodextrin or derivatives thereof, and an acidic buffer to adjust pH=3.5-6.0,

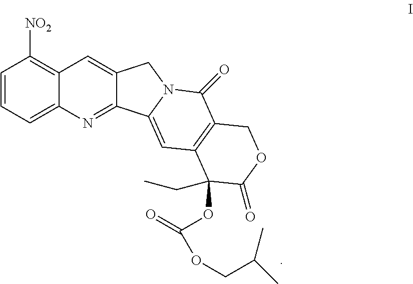 [SMILES: CC[C@@]1(OC(=O)OCC(C)C)C(=O)OCc2c1cc1n(c2=O)Cc2cc3c([N+](=O)[O-])cccc3nc2-1]

2. The pharmaceutical composition according to the claim 1, characterized in that the weight ratio of camptothecin compound of formula I and β-cyclodextrin or derivatives thereof is preferably 1:200 to 1:800.

3. The pharmaceutical composition according to the claim 1, characterized in that the β-cyclodextrin or derivatives thereof are selected from the group consisting of hydroxypropyl-β-cyclodextrin and/or sulfobutyl ether sodium β-cyclodextrin.

4. The pharmaceutical composition according to the claim 1, characterized in that the acidic buffer is selected from the group consisting of tartaric acid or salts thereof, citric acid or salts thereof, hydrochloric acid or salts thereof, acetic acid or salts thereof, maleic acid or salts thereof, malic acid or salts thereof, sulphuric acid or salts thereof, phosphoric acid or salts thereof, lactic acid or salts thereof.

5. The pharmaceutical composition according to the claim 1, characterized in that the dosage form of the pharmaceutical composition is a pharmaceutical composition in aqueous solution form or in solid form.

6. The pharmaceutical composition according to the claim 5, characterized in that the pH of the pharmaceutical composition in aqueous solution form is adjusted by the acidic butter in the range of 3.5˜5.5, or adjust the pH of the pharmaceutical composition in solid form dissolved after added water in the range of 3.5˜6.0.

7. A method for preparing the pharmaceutical composition according to the claim 1 including the following steps:
a water-soluble β-cyclodextrin or derivatives thereof is dissolved in water, addition of an acidic butter to adjust pH in the range of 3.5 to 6.0; then the compound of formula I is mixed into and stirred to dissolve; addition of medicinal activated C, stirring for adsorption and decarburization; and addition of water to the full volume, sterilization filtration, spray drying or freeze drying after split charging, to give the pharmaceutical composition, or directly split charging.

8. Use of the pharmaceutical composition according to claim 1 for preparing the treatment of the solid tumour medicament.
